(19) United States
(12) Patent Application Publication
(10) Pub. No.: US 2010/0055190 A1
(43) Pub. Date: Mar. 4, 2010

(54) Sterol-Containing Compositions

(75) Inventors: Peter Horlacher, Bellenberg (DE); Dieter Hietsch, Illertissen (DE); Wolfgang Albiez, Illertissen (DE); Franz Timmermann, Illertissen (DE); Katja Beck, Bellenberg (DE)

Correspondence Address: FOX ROTHSCHILD LLP, 997 Lenox Drive, Bldg. #3, Lawrenceville, NJ, 08648, US

(73) Assignee: Cognis IP Management GmbH, Duesseldorf (DE)

(21) Appl. No.: 12/282,171

(22) PCT Filed: Feb. 27, 2007
(86) PCT No.: PCT/EP07/01657

§ 371 Date: Feb. 10, 2009

(30) Foreign Application Priority Data
Mar. 8, 2006 (DE) 10 2006 011 090.0

(51) Int. Cl. A61K 9/14; A61K 31/56; A61P 9/10

(52) U.S. Cl. 424/489; 514/182

(57) ABSTRACT

The invention relates to a powder formulation containing sterol in the form of a super-cooled melt from sterols and sterol esters, provided that the sterol ester content is at least 30 wt % in relation to the content of the powder. The introduced sterol ester is a fatty acid ester having a chain length of 2 to 24, preferably 8 to 12 carbon atoms. As the sterol preparations are wettable and melt easily without the use of complex equipment, they can easily be incorporated into food, and produce, in particular, good organoleptic and sensory properties, in particular, in drinks.

### CROSS REFERENCE TO RELATED APPLICATIONS

This application is the National Phase entry of PCT/EP2007/001657, filed Feb. 27, 2007, which claims priority to German patent application number DE 10 2006 011 090.0, filed Mar. 8, 2006, which are incorporated herein by reference in their entireties.

### FIELD OF THE INVENTION

This invention relates generally to powder compositions and, more particularly, to compositions for incorporation in foods, and to cosmetic and pharmaceutical preparations containing mixtures of sterols and/or stanols with esters thereof in the form of a supercooled melt, to a process for their production and to preparations, more particularly foods, containing these formulations.

### BACKGROUND OF THE INVENTION

The literature offers numerous formulation options for enabling poorly soluble phytosterols and phytostanols, which are known to lower cholesterol, to be incorporated in food preparations, cosmetics or pharmaceutical products. Besides leading to poor dispersibility, the unfavorable solubility behavior of the substances reduces their bioavailability and adversely affects the stability of the food preparations. The literature describes how the availability of sterols can be improved by reducing the particle sizes, mainly by micronization. However, the reduction of particle size and the resulting surface enlargement in turn give rise to poor processability because the energy-rich particles aggregate and show very poor wettability.

Accordingly, it is generally necessary to use emulsifiers which distinctly improve the dispersion properties. Even though food emulsifiers are distinguished by good compatibility and have already been known for some time, efforts are being made to reduce the quantity of emulsifiers or even to avoid them altogether because emulsifiers can also influence the bioavailability of other substances present in the foods or can adversely affect the stability of the formulations.

In addition, the incorporation of emulsifiers still requires further technically imaginative formulation developments to minimize the disadvantages of poor further processing. Thus, European patent EP1 005 859 B1, U.S. Pat. No. 6,267,963 B1 and International patent application WO 03/77680 A1 propose sterol formulations which are produced by melting sterols and emulsifiers together. These sterol/emulsifier complexes, which crystallize out together, enable the sterols to be easily and directly incorporated in food preparations. However, the reduced sterol content of the formulations has a negative effect because the increase in the quantities used also increases the input of emulsifiers.

An alternative to the pure sterols or stanols is to use derivatives esterified with fatty acids because sterol esters are comparable with the sterols in their cholesterol-lowering effect. The esterified derivatives are a little easier to incorporate because, by virtue of their lower melting point, they can be homogeneously distributed by melting in the heated foods. Commercially available sterol esters are generally derivatives with fatty acids from vegetable oils such as, for example, sunflower oil, rapeseed oil, linseed oil, rice bran oil, safflower oil or soybean oil. They are produced by transesterification or esterification of free sterols, such as by the process disclosed in European patent EP 0 914 329 B1. The choice of the fatty acids influences the properties of the various sterol derivatives in regard to melting point, stability and solubility, as already shown in U.S. Pat. No. 3,751,569 and in European patent EP 1 075 191 B1. One disadvantage of the lower melting point of the sterol esters lies in their physico-chemical stability. Applicants have come to recognize that sterol fatty acid esters are difficult to process to powders because they have a tacky paste-form or viscous consistency, depending on the fatty acid used.

Accordingly, the present invention solves one or more of the aforesaid problems by providing compositions was to enable unesterified sterols and/or stanols to be readily and uniformly dispersed and incorporated in foods, while at the same time preferably providing for favorable sensory and organoleptic properties in the foods.

### DETAILED DESCRIPTION OF PREFERRED EMBODIMENTS

The present invention relates to sterol-containing compositions, preferably in the form of a supercooled melt of sterols and sterol esters wherein the sterol ester content is at least about 30% by weight, based on the weight of the composition. As used herein, the term “sterol” includes within its meaning not only sterols but also sterol-derived compounds, such as stanols, which are formed by hydrogenation of sterols.

The compositions according to the invention can be obtained by melting free unesterified sterols and sterol esters together, solidifying the molten mixture by rapid cooling and grinding the solidified melt, preferably to produce a powder.

From their production, commercially available sterol esters contain a more or less large percentage of free sterols owing to the fact that the sterols present are not completely esterified. European patent EP 0 911 385 B1, for example, describes sterol esters with a degree of esterification of 40 to 85%.

Although the supercooled melt of both products—the free sterols and the esterified sterols—according to the invention contains the same molecules, it has totally different properties to the partially esterified sterols and the pure powder mixture of both components because of its different physico-chemical constitution. All the products are easy to differentiate by differential scanning calorimetry (DSC). Surprisingly, the preferred sterol compositions according to the invention have improved organoleptic properties in relation to pure sterols and sterol esters. In contrast to pure sterol esters, they can be processed to free-flowing powders which then have a considerably higher total sterol content. The preferred powders according to the invention contain at least about 65% by weight, preferably at least about 75% by weight and, more preferably in certain embodiments at about 80% by weight total sterol, based on the weight of the powder.

Sterols obtained from plants and vegetable raw materials—so-called phytosterols and phytostannols—are preferably used in the present invention. Known examples are ergosterol, brassica sterol, campesterol, avenasterol, desmosterol, clionasterol, stigmasterol, poriferasterol, chalinosterol, sitosterol and mixtures thereof. Of these, β-sitosterol and campesterol are preferably used. Hydrogenated saturated forms of the sterols, known as stanols, are also included among the compounds used. Again, β-sitostanol and campestanol are preferred. Vegetable raw material sources include inter alia seeds and oils of soybeans, canola, palm kernels, corn, coconut, rape, sugar cane, sunflower, olive, cotton, soya, peanut or products from the production of tall oil.

Applicants have found that, in general, the larger the quantity of pure sterol used in the supercooled melt, the higher the melting range of the formulation. A fine-particle powder with good flow properties can be produced. In certain embodiments a melting point below about 120° C. is advantageous because it provides for melting in the food formulation and hence for easy processing. The particle size of the sterol powder to be processed can then be above the micron range. However, the quantity of the unesterified sterol used in the melt is thus also limited by the melting point of the supercooled melt. In order still to aid good processability by simple melting of the formulation in the food, the quantity of sterol esters used preferably in not below about 30% by weight. A ratio by weight of sterol to sterol ester of from about 3:7 to about 7:3 and preferably in the range from about 1:4 to about 3:2 is advantageous.

The preferred sterol esters are normally fatty acid esters with fatty acids having chain lengths of 2 to 26 carbon atoms. Possible fatty acids are short-chain fatty acids, such as acetic acid, propionic acid, butyric acid, caproic acid, caprylic acid, capric acid, dicarboxylic acids such as, for example, oxalic acid and maleic acid, hydroxy acids, such as citric acid, lactic acid, and long-chain saturated and unsaturated fatty acids, such as lauric acid, myristic acid, palmitic acid, stearic acid, arachidonic acid, behenic acid, oleic acid, erucic acid, elaidic acid, linoleic acid, conjugated linoleic acid, linolenic acid, docosahexaenoic acid and eicosapentaenoic acid. Sterol esters of saturated and unsaturated fatty acids having chain lengths of 16 and 18 carbon atoms, which are obtained, for example, by esterification with sunflower and rapeseed oil fatty acids, are preferred.

Saturated fatty acids with a chain length of 6 to 10 carbon atoms in a distribution as present in the form of the medium-chain triglycerides (MCTs)—also known as Miglykol®—esterified with glycerol are particularly preferred. Sterol esters with fatty acids of this chain length generally have good organoleptic properties and are generally substantially more stable to oxidation than commercially available sterol esters with long-chain unsaturated fatty acids. Since MCT oils in the field of human nutrition reduce the uptake of fats and increase both the burning of fats and the metabolism rate, sterol esters of these fatty acids could also produce other main and secondary effects of value in terms of nutrition physiology.

Regarding the choice of medium-chain fatty acids, chain lengths of 8 and 10 carbon atoms are preferably used in a fatty acid distribution (ratio by weight) of C8 to C10 of from about 100:0 to about 0:100, preferably from about 60:40 to about 50:50 and, more particularly, from about 75:25 to about 65:35. The fatty acid mixture preferably contains at most about 7% by weight, preferably at most about 5% by weight and, more particularly, at most about 3% by weight of other chain lengths. A sterol ester with medium-chain fatty acids having a chain length distribution of from about 69 to about 75% by weight C8 and from about 23 to about 27% by weight C10 (see Example) has improved stability in relation to commercially available fatty acid esters with sunflower and rapeseed oil fatty acids (mainly linoleic acid, oleic acid and small quantities of palmitic and stearic acid) and improved organoleptic properties and improved processability in relation to pure sterol.

In contrast to the usual sterol esters based on unsaturated fatty acids, such as sunflower oil fatty acid or rapeseed oil fatty acid, the mixtures of medium-chain sterol esters and sterols also show high stability to oxidation. The sensory properties are not impaired by the usual storage and/or transportation conditions. The product according to the invention can be stored under standard conditions (RT) and can even be transported at elevated temperatures, as encountered in Asiatic countries and/or in summer (30-40° C.), without any damage to its properties.

The sterol composition powder according to the invention is preferably powder with a high total sterol content which enables the lipophilic active components to be readily incorporated in foods, more particularly beverages. The preferred powder shows little tendency to agglomerate and, hence, has good flow properties. It is distinguished by good homogeneity and, by virtue of its improved wettability, can be further processed without major outlay on machinery. In addition, it is uniformly distributed very quickly in the final formulation.

Production can be carried out by standard methods, for example by esterification of sterol and/or mixtures of various sterols or stanols with edible fatty acids, preferably with saturated medium-chain C₈ and C₁₀ fatty acids. Esters of the fatty acids can also be esterified. Corresponding processes are known and available.

The selected sterol esters are preferably melted together with the free sterols at from about 120° C. to about_190° C. and rapidly cooled to a temperature of from about 10° C. to about_25° C. The melt thus solidified is preferably size-reduced with simple impact tools and mills, preferably at low temperatures. Cold spraying processes known to the expert may also be used for size reduction to the desired particle size distribution. Very different particle size distributions can be adjusted according to the process used and the further processing intended. An average particle size of at most 3 mm, preferably between 1 μm and 1 mm, more preferably below 100 μm and most preferably below 15 μm has proved to be preferred in certain embodiments.

The powder according to the invention, when incorporated in foods, eliminates the need to use highly surface-active emulsifiers, such as—in particular—lecithins, monoglycerides, diglycerides, polysorbates, sodium stearyl lactylate, glycerol monostearate, lactic acid esters and polyglycerol esters, propylene glycol esters, polyoxyethylene esters, diacetyl succinic acid esters.

The sterol-containing formulations produced by this process may readily be incorporated in foods, more particularly in milk, milk beverages, whey and yogurt beverages, margarine, fruit juices, fruit juice mixtures, fruit juice beverages, vegetable beverages, still and sparkling beverages, soya milk beverages and protein-rich liquid food substitute beverages and fermented milk preparations, yogurt, drinking yogurt, or cheese preparations, cereals and nutrition bars, and also in cosmetic or pharmaceutical preparations.

Accordingly, the present invention also relates to food preparations containing sterol formulations with the composition mentioned above. They are preferably used in beverages and milk products which then contain 0.1 to 50% by weight and preferably 1 to 20% by weight of the compositions of the present invention, based on the total weight of the foods.

Production can be carried out by standard methods, for example by esterification of sterol and/or mixtures of various sterols or stanols with saturated medium-chain C₈ and C₁₀ fatty acids. Esters of the fatty acids can also be esterified by transesterification Esterification with the corresponding anhydrides or acid halides is also possible. Corresponding processes are known and available.

The sterol fatty acid ester according to preferred aspects of the invention eliminates the need for emulsifiers when it comes to incorporation in foods. It may readily be incorporated in foods selected from the group consisting of spreading fats, margarine, butter, vegetable oils, frying fats, peanut butter, mayonnaise, dressings, cereals, bread and confectionery, cakes, wheat bread, rye bread, toast, crispbread, ice cream, puddings, milk products, yogurt, cottage cheese, cream, sweets, chocolate, chewing gum, muesli bars, milk beverages, soya beverages, fruit juices, vegetable juices, fermented beverages, noodles, rise, sauces, cheese, spreading cheese, meat and sausages.

### EXAMPLES

### Example 1

### Production of the Sterol Ester with Medium-Chain Fatty Acids

726 g of a fatty acid containing 8 to 10 carbon atoms (69-75% C₈, 23-27% C₁₀) were introduced into a reaction vessel and heated under nitrogen to 120° C. 1120 g tall oil sterol and 480 g rape sterol were then slowly added in three portions, the temperature being kept above 100° C. The reactor contents were then heated for 3 hours to 210° C., the upper phase of the reaction distillate being continuously returned to the reaction mixture. The mixture was then evacuated to 100 mbar and stirred for 4 hours. The excess fatty acid was then distilled off at 15 mbar and the reaction mixture was cooled to 90° C. and purged with nitrogen. The mixture was dried for 30 minutes at 85° C./<30 mbar before purging with nitrogen. The concluding purification step was carried out at 190° C./3 mbar by introduction of stripping steam (0.2 g per minute). 1911 g of an odorless, light, sensorially neutral, high-melting solid and 16 g of a yellow clear distillate were obtained as residue.

### Example 2

### Production of the Powdered Supercooled Melts of Sterols and Sterol Esters

a) 83.3 g sterol esters with C₈₋₁₀ fatty acids (from Example 1) were melted together with 11.7 g tall oil sterol and 5.0 g rape sterol at 140±3° C. A clear melt was formed, appearing clear up to a temperature of 80° C. The melt was poured out onto a metal plate at room temperature and, 20 hours after solidifying, was ground in a Krups beater mill. Taking into account the residual content of esterified sterols in the free sterol used and free sterol in the sterol ester used, the resulting ground powder in the form of the supercooled melt had a total sterol content in a ratio to free sterol of 81.7% by weight to 20.4% by weight.
b) 62.5 g sterol esters with C₈₋₁₀ fatty acids were melted together with 26.3 g tall oil sterol and 11.3 g rape sterol at 140±3° C. The melt was poured out onto a metal plate at room temperature, crystallized more quickly than the melt of test a) and, 20 hours after solidifying, was ground in a Krups beater mill. The ground powder had a total sterol content in a ratio to free sterol of 85.7% by weight to 39.8% by weight.
c) The test described in a) and b) was repeated with 41.7 g sterol esters with C₈₋₁₀ fatty acids, 40.8 g tall oil sterol and 17.5 g rape sterol. The melt was clear up to a temperature of 100° C., but crystallized out much more quickly than a) and b). The ground powder had a total sterol content in a ratio to free sterol of 89.8% by weight to 59.2% by weight.

An increased amount of free unesterified sterols increases the solidification temperature of the supercooled melt and, accordingly, is also of advantage to the behavior of the ground melt. Powder a) produced a slightly tacky powder while powder b) was less tacky; variant c) had the best flow properties.

However, if the melting range of the formulation according to the invention is greatly increased by addition of an increased amount of free sterol, the powder can only be further processed in water-based formulations by very fine grinding. A melting range below 100° C. is advantageous for incorporation in beverages because corresponding formulations can be introduced into the heated foods after melting and allow easy and simple further processing and uniform distribution in the end consumer product.

### Dispersion Test

The powder thus obtained was dispersed in milk and water in comparison with ground sterols having a comparable particle size distribution. To this end, ca. 250 ml of the liquid to be tested were poured into a glass beaker and stirred (ca. 100 r.p.m.). 2.5 g of the respective powders were added to the stirred liquid and dispersion behavior was evaluated.

All the powders according to the invention could be uniformly dispersed in cold water (15° C.), in hot water (60° C.) and in milk (18° C.) whereas the untreated free ground sterol was poorly dispersed and, due to the hydrophobic surface, remained on the surface of the liquid. Sensorially, major differences were found. The powders according to the invention tasted neutral in water and milk and did not form a coating on the gums and oral mucous membrane whereas the untreated powder stuck to the oral mucous membrane and, in addition to a typical negative sterol taste, left behind an unpleasant sensory impression.

1-11. (canceled)

12. A composition comprising sterol-containing powder in the form of a supercooled melt of sterols and sterol esters, provided that the sterol ester content is at least 30% by weight of the total sterol content.

13. The composition of claim 12, wherein the sterol ester comprises a fatty acid ester with chain lengths of 2 to 24 carbon atoms.

14. The composition of claim 13, wherein the sterol ester comprises a fatty acid ester with chain lengths of 6 to 10 carbon atoms.

15. The composition of claim 12, wherein the total sterol content is at least 65% by weight, based on the weight of the composition.

16. The composition of claim 14, wherein the total sterol content is at least 65% by weight, based on the weight of the composition.

17. The composition of claim 14, wherein the total sterol content is at least 65% by weight, based on the weight of the composition.

18. The composition of claim 12, wherein the ratio by weight of sterol to sterol ester is in the range from about 3:7 to about 7:3.

19. The composition of claim 12, wherein the ratio by weight of sterol to sterol ester is in the range from about 1:4 to about 3:2.

20. The composition of claim 12, wherein the average particle size is at most 3 mm.

21. A food preparation comprising about 0.1 to about 50% by weight of the composition of claim 12.

22. A beverage or milk product comprising about 0.1 to about 50% by weight of the composition of claim 12.

23. A process for the production of sterol-containing powder compositions, comprising
(a) melting a mixture of sterols and/or stanols and sterol and/or stanol esters together at a temperature from about 120° C. to about 190° C. to form a melt,
(b) cooling said melt to form a solid, and
(c) grinding said solid to form a powder.
